(19) United States
(12) Patent Application Publication
(10) Pub. No.: US 2024/0192239 A1
(43) Pub. Date: Jun. 13, 2024

(54) SAMPLE ANALYSIS DEVICE, SAMPLE ANALYSIS METHOD, PHARMACEUTICAL ANALYSIS DEVICE AND PHARMACEUTICAL ANALYSIS METHOD

(71) Applicant: SHIMADZU CORPORATION, Kyoto-shi (JP)

(72) Inventor: Yusuke NAKAI, Kyoto-shi (JP)

(21) Appl. No.: 18/517,981

(22) Filed: Nov. 22, 2023

(30) Foreign Application Priority Data
Nov. 25, 2022 (JP) 2022-188548

(51) Int. Cl. G01N 35/00

(52) U.S. Cl. CPC G01N 35/00693

(57) ABSTRACT

A sample analysis device includes a quantitative information calculator that calculates, based on a plurality of measurement data pieces, a plurality of quantitative measurement information pieces of a substance included in a sample, and an estimator that provides the plurality of quantitative measurement information pieces calculated by the quantitative information calculator to a reaction model to estimate a parameter of the reaction model. In the reaction model, an integrated error term for integration of errors based on setting values of the temperature and the humidity set under the plurality of analysis conditions is included in the reaction model.

### BACKGROUND

### Technical Field

The present invention relates to a sample analysis device and a sample analysis method for analyzing a substance included in a sample, and a pharmaceutical analysis device and a pharmaceutical analysis method for analyzing an active ingredient, an impurity or the like included in a formulation or the like.

### Description of Related Art

Accelerated testing is performed on a product such as a formulation, for example. In this test, a product is stored under a condition severer than a normal storage condition. After a predetermined period of time has elapsed, a component analysis of the product is performed with use of an analysis device. Thus, it is possible to perform, in a short period of time, a component analysis test of the product which originally requires long-term storage or to evaluate variations in result of component analysis. When the components of the product after long-term storage are analyzed based on a measurement result obtained in the accelerated testing, a reaction model is estimated with use of an Arrhenius equation or a modified Arrhenius equation. In the technical note for determination of a reaction model formula based on DSC data by AKTS/Thermokinetics, which is found on http://www.palmetrics.co.jp/_userdata/TKTS_07_2019R.pdf, a method of performing lifetime estimation based on accelerated testing data is presented.

### SUMMARY

In the accelerated testing, acceleration factors such as a temperature and a humidity are set to values severer than those for a normal storage condition, for example. However, depending on a testing environment, an error may be generated between actual temperature and humidity and their set values. This error is a factor that degrades accuracy of estimation of a reaction model.

An object of the present invention is to suppress degradation in accuracy of estimation of a reaction model due to an error caused by an acceleration factor.

A sample analysis device according to one aspect of the present invention includes an acquirer that acquires a plurality of measurement data pieces obtained by an analysis of a sample using an analysis device under a plurality of analysis conditions, the analysis conditions including a temperature and a humidity as acceleration factors, a quantitative information calculator that calculates, based on the plurality of measurement data pieces, a plurality of quantitative measurement information pieces of a substance included in the sample, an estimator that retrieves a reaction model stored in a storage device, models quantitative estimation information of the substance with use of the reaction model, and provides the plurality of quantitative measurement information pieces calculated by the quantitative information calculator to the reaction model to estimate a parameter of the reaction model, and a calculator that calculates, based on the parameter estimated by the estimator, quantitative estimation information of the substance at an arbitrary point in time or calculates information in regard to a period of time until quantitative estimation information of the substance reaches a predetermined threshold value, wherein an integrated error term for integration of errors based on setting values of the temperature and the humidity set under the plurality of analysis conditions is included in the reaction model.

A sample analysis device according to another aspect of the present invention includes an acquirer that acquires a plurality of measurement data pieces obtained by an analysis of a sample using an analysis device under a plurality of analysis conditions, the analysis conditions including a temperature and a humidity as acceleration factors, a quantitative information calculator that calculates, based on the plurality of measurement data pieces, a plurality of quantitative measurement information pieces of a substance included in the sample, an estimator that retrieves a reaction model stored in a storage device, models quantitative estimation information of the substance with use of the reaction model, and provides the plurality of quantitative measurement information pieces calculated by the quantitative information calculator to the reaction model to estimate a parameter of the reaction model, and a calculator that calculates, based on the parameter estimated by the estimator, quantitative estimation information of the substance at an arbitrary point in time or calculates information in regard to a period of time until quantitative estimation information of the substance reaches a predetermined threshold value, wherein an additional reaction is set in the reaction model in accordance with an initial value.

A sample reaction device according to another aspect of the present invention includes an acquirer that acquires a plurality of measurement data pieces obtained by an analysis of a sample using an analysis device under a plurality of analysis conditions, the analysis conditions including a temperature and a humidity as acceleration factors, a quantitative information calculator that calculates, based on the plurality of measurement data pieces, a plurality of quantitative measurement information pieces of a substance included in the sample, an estimator that retrieves a reaction model stored in a storage device, models quantitative estimation information of the substance with use of the reaction model, and provides the plurality of quantitative measurement information pieces calculated by the quantitative information calculator to the reaction model to estimate a parameter of the reaction model, and a calculator that calculates, based on the parameter estimated by the estimator, quantitative estimation information of the substance at an arbitrary point in time or calculates information in regard to a period of time until quantitative estimation information of the substance reaches a predetermined threshold value, wherein a time difference in regard to start of an analysis is set in the reaction model.

The present invention is also directed to a sample analysis method, a pharmaceutical analysis device and a pharmaceutical analysis method.

Other features, elements, characteristics, and advantages of the present disclosure will become more apparent from the following description of preferred embodiments of the present disclosure with reference to the attached drawings.

### BRIEF DESCRIPTION OF THE DRAWING

FIG. 1 is a diagram showing the configuration of a sample analysis device according to the present embodiment;

FIG. 2 is a block diagram showing the functions of the sample analysis device according to the present embodiment;

FIG. 3 is a diagram showing the contour of a log likelihood;

FIG. 4 is a diagram showing the cross section of the contour taken along the line A-B in FIG. 3;

FIG. 5 is a graph showing a change in peak area ratio in a case in which no additional reaction occurs;

FIG. 6 is a graph showing a change in peak area ratio in a case in which an additional reaction occurs;

FIG. 7 is a diagram showing a difference in estimated shelf-life;

FIG. 8 is a diagram obtained when an additional reaction is modeled by a function;

FIG. 9 is a diagram obtained when an offset Δt to t=0 is modelled;

FIG. 10 is a flowchart showing a sample analysis method according to an embodiment;

FIG. 11 is a flowchart showing the sample analysis method according to the embodiment;

FIG. 12 is a flowchart showing the sample analysis method according to the embodiment;

FIG. 13 is a diagram showing the simulation data of a peak area ratio;

FIG. 14 is a diagram showing an estimation result obtained with use of a model formula of the formula (6); and

FIG. 15 is a diagram showing an estimation result obtained with use of a model formula into which a hierarchical error is introduced.

### DETAILED DESCRIPTION

A sample analysis device, a sample analysis method, a pharmaceutical analysis device and a pharmaceutical analysis method according to embodiments of the present invention will now be described with reference to the attached drawings.

### (1) Configuration of Sample Analysis Device

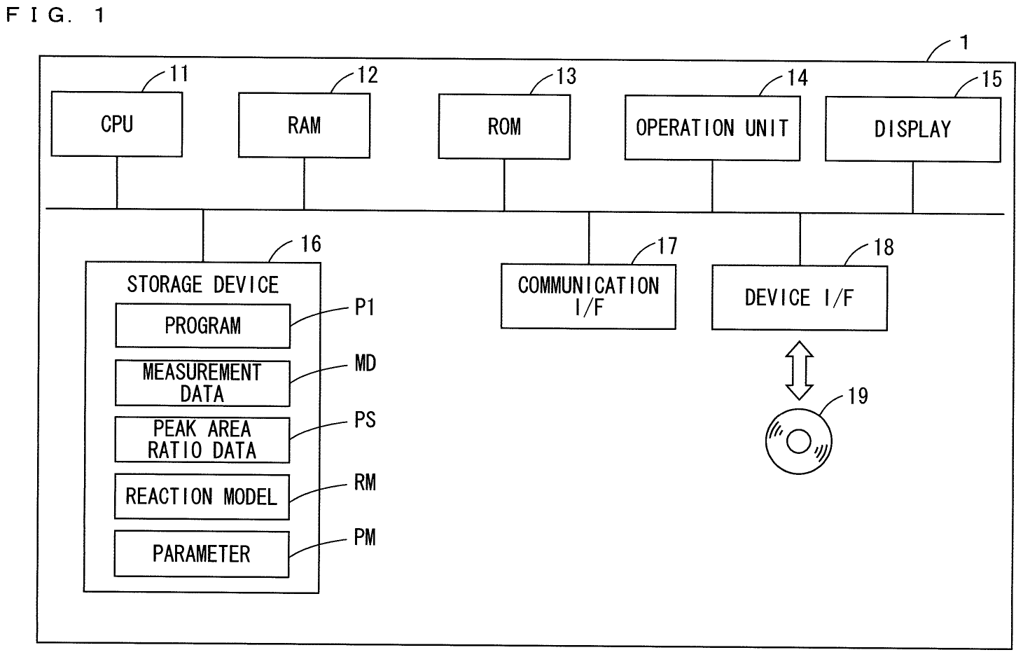

FIG. 1 is a diagram showing the configuration of the sample analysis device 1 according to an embodiment. The sample analysis device 1 of the present embodiment acquires measurement data MD of a sample obtained in an analysis device such as a liquid chromatograph, a gas chromatograph or a mass spectrometer. In the present embodiment, in particular, the sample analysis device 1 is used as a pharmaceutical analysis device that analyzes a pharmaceutical (a formulation or a drug substance) as a sample, by way of example.

The sample analysis device 1 of the present embodiment is constituted by a personal computer. As shown in FIG. 1, the sample analysis device 1 includes a CPU (Central Processing Unit) 11, a RAM (Random Access Memory) 12, a ROM (Read Only Memory) 13, an operation unit 14, a display 15, a storage device 16, a communication interface (I/F) 17 and a device interface (I/F) 18.

The CPU 11 controls the sample analysis device 1 as a whole. The RAM 12 is used as a work area for execution of a program by the CPU 11. Various data, a program and the like are stored in the ROM 13. The operation unit 14 receives an input operation performed by a user. The operation unit 14 includes a keyboard, a mouse, etc. The display 15 displays information such as a result of analysis. The storage device 16 is a storage medium such as a hard disc. A program P1, measurement data MD, peak area ratio data PS, a reaction model RM (data defining a reaction model function) and a parameter PM are stored in the storage device 16.

The program P1 models the quantitative estimation information of a substance included in a sample by using the reaction model RM retrieved from the storage device 16. Further, the program P1 provides the quantitative measurement information of a plurality of substances to the reaction model RM to estimate the parameter PM of the reaction model RM. Further, the program P1 calculates the quantitative estimation information of a substance at an arbitrary point in time based on the estimated parameter PM. Further, the program P1 calculates, based on the estimated parameter PM, the information in regard to a period of time until the quantitative estimation information of the substance reaches a predetermined threshold value.

The communication interface 17 is an interface that communicates with another computer through wireless or wired communication. The device interface 18 is an interface that accesses a storage medium 19 such as a CD, a DVD or a semiconductor memory.

### (2) Functional Configuration of Sample Analysis Device

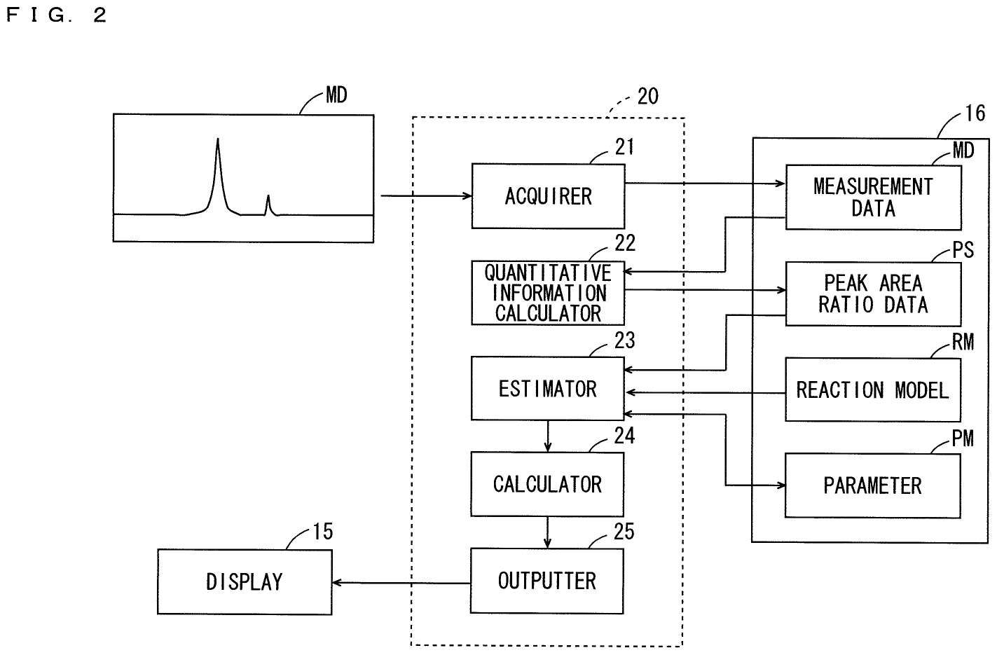

FIG. 2 is a block diagram showing the functional configuration of the sample analysis device 1. In FIG. 2, a controller 20 is a function that is implemented by execution of the program P1 by the CPU 11 while the CPU 11 uses the RAM 12 as a work area. The controller 20 includes an acquirer 21, a quantitative information calculator 22, an estimator 23, a calculator 24 and an outputter 25. That is, the acquirer 21, the quantitative information calculator 22, the estimator 23, the calculator 24 and the outputter 25 are the functions implemented by execution of the program P1. In other words, each of the functions 21 to 25 is a function included in the CPU 11.

The acquirer 21 receives measurement data MD. The acquirer 21 receives the measurement data MD from an analysis device such as a liquid chromatograph, a gas chromatograph or a mass spectrometer, or another computer via the communication interface 17, for example. Alternatively, the acquirer 21 receives the measurement data MD stored in the storage medium 19 via the device interface 18. The measurement data MD acquired by the acquirer 21 is multidimensional data acquired by a multidimensional detector included in the chromatograph, for example. The measurement data MD is three-dimensional data having a retention-time direction, a spectral direction (frequency direction) and an intensity as elements, by way of example. In this case, the measurement data MD is represented as matrix data having the row corresponding to the retention-time direction, the column corresponding the spectral direction and the intensity as an element. For example, the measurement data MD is the data acquired in a liquid chromatograph including a PDA detector (photodiode array detector). The acquirer 21 stores the acquired measurement data MD in the storage device 16.

The quantitative information calculator 22 calculates peak area ratio data PS based on the measurement data MD retrieved from the storage device 16. The peak area ratio data PS is an example of “quantitative measurement information of a substance included in a sample” in the present invention. In the present embodiment, as the quantitative measurement information of a substance included in a sample, the ratio of a peak area of impurities with respect to a peak area of an active ingredient included in a pharmaceutical is used, by way of example. The quantitative information calculator 22 stores the calculated peak area ratio data PS in the storage device 16.

Here, the measurement data MD acquired by the acquirer 21 includes a plurality of data pieces obtained by an analysis of a sample under a plurality of analysis conditions in an analysis device such as a liquid chromatograph. Therefore, the quantitative information calculator 22 calculates a plurality of peak area ratio data pieces PS corresponding to a plurality of measurement data pieces MD. In particular, in the present embodiment, the measurement data MD acquired by the acquirer 21 is the data obtained under a plurality of analysis conditions in which a temperature and a humidity are acceleration factors. Therefore, the plurality of peak area ratio data pieces PS calculated by the quantitative information calculator 22 is the data obtained under the plurality of analysis conditions in which the temperature and humidity are acceleration factors.

The estimator 23 uses the reaction model RM retrieved from the storage device 16 to model the quantitative estimation information of a substance included in a sample. In this example, the estimator 23 uses the reaction model RM to model a peak area ratio (a ratio of a peak area of impurities with respect to a peak area of an active ingredient included in a pharmaceutical) as “quantitative estimation information of a substance.” The estimator 23 provides the peak area ratio data PS (quantitative measurement information of a substance) calculated by the quantitative information calculator 22 to the reaction model RM to estimate a parameter PM of the reaction model RM. The estimator 23 stores the estimated parameter PM in the storage device 16.

The calculator 24 calculates, based on the parameter PM estimated by the estimator 23, the estimation information of a peak area ratio (the quantitative estimation information of a substance) at an arbitrary point in time. The quantitative estimation information includes a quantitative value, a confidence interval or a quantile of the substance at an arbitrary point in time. Further, the calculator 24 calculates, based on the parameter PM estimated by the estimator 23, the information in regard to a period of time until the estimation information of a peak area ratio (the quantitative estimation information of a substance) reaches a predetermined threshold value. The information in regard to a period of time includes a period of time, a confidence interval or a quantile until the quantitative estimation information of a substance reaches the predetermined threshold value.

The outputter 25 causes quantitative estimation information of a substance to be displayed on the display 15. Further, the outputter 25 causes the information in regard to a period of time until the quantitative estimation information of a substance reaches a predetermined threshold value to be displayed on the display 15.

The program P1 is stored in the storage device 16, by way of example. In another embodiment, the program P1 may be provided in the form of being stored in the storage medium 19. The CPU 11 may access the storage medium 19 via the device interface 18 and may store the program P1 stored in the storage medium 19 in the storage device 16 or the ROM 13. Alternatively, the CPU 11 may access the storage medium 19 via the device interface 18 and may execute the program P1 stored in the storage medium 19. Alternatively, the CPU 11 may download the program P1 stored in a server on a network via the communication interface 17.

### (3) Estimation Processing

### (3-1) Basic Concept of Estimation Processing

Prior to description of the content of the estimation processing executed by the estimator 23, the basic concept of the estimation processing will be described. It is possible to model a peak area ratio β (t, T, H) obtained when a sample is stored at an absolute temperature T [K] and a relative humidity H [%] for a period t [day] by using a combination of a modified Arrhenius equation and a solid reaction model equation. Here, the peak area ratio β is the ratio of a peak area of impurities with respect to a peak area of an active ingredient included in a pharmaceutical, by way of example. First, the modified Arrhenius equation is expressed by the formula (1).

$$[ Formula 1 ]$$

$$k ( T , H ) = A exp ( - \frac{E}{RT} + BH ) \qquad ( 1 )$$

In the formula (1), R represents a gas constant (≈8.314 J/(K·mol)), A represents a frequency factor, E represents an activation energy and B represents a parameter in regard to humidity. While there are various solid reaction models, a model such as the formula (2) using a reaction progress rate α(t) and parameters m and n is considered as an example.

$$[ Formula 2 ]$$

$$\frac{d α ( t )}{dt} = k \cdot {( α ( t ) )}^{m} {( 1 - α ( t ) )}^{n} \qquad ( 2 )$$

With use of a peak area ratio β_{∞} obtained when t=∞, the relationship between the peak area ratio β(t) and the reaction progress rate α(t) can be expressed as in the formula (3).

[Formula 3]

β(t)=α(t)·β_{∞} (3)

When the formula (2) is substituted into the formula (3), the following formula (4) is obtained.

$$[ Formula 4 ]$$

$$\frac{d β ( t , T , H )}{dt} = k ( T , H ) \cdot {β}_{∞} {( \frac{β ( t , T , H )}{{β}_{∞}} )}^{m} {( 1 - \frac{β ( t , T , H )}{{β}_{∞}} )}^{n} \qquad ( 4 )$$

When the initial value of the peak area ratio β(t, T, H) is β₀, and the formula (4) and the formula (1) are combined, the peak area ratio β(t, T, H) for any value for each of t, T and H is expressed by the formula (5).

$$[ Formula 5 ]$$

$$β ( t , T , H ) = {\int }_{0}^{t} k ( T , H ) \cdot {β}_{∞} {( \frac{β ( {t}^{′} , T , H )}{{β}_{∞}} )}^{n} {( 1 - \frac{β ( {t}^{′} , T , H )}{{β}_{∞}} )}^{n} {dt}^{′} + {β}_{0} \qquad ( 5 )$$

$$where$$

$$k ( T , H ) = A exp ( - \frac{E}{RT} + BH )$$

A, E, B, m, n, β₀ and β_{∞} are parameters for the model formula for the peak area ratio β(t). When a value is provided to each parameter, β(t, T, H) is obtained by integral calculation. This integral calculation may be performed approximately.

The parameters A, E, B, m, n, β₀ and β_{∞} in the formula (5) can be estimated by measurement of L peak area ratios {β(t₁, t₁, H₁), β(T₂, T₂, H₂), . . . , β(t_{L}, T_{L}, H_{L})} stored for a period of tᵢ at an absolute temperature Tᵢ and a relative humidity Hᵢ and use of a regression technique such as a least squares method, MAP estimation or Bayesian inference. Thus, the peak area ratio β with respect to any value for each of t, T and H can be estimated. Practically, the temporal change of a peak area ratio β under long-term storage conditions of 25° C. 60% RH is often calculated to obtain the number of days until the peak area ratio β exceeds a predetermined threshold value.

However, with Bayesian inference, it is also necessary to explicitly model an error and provide a prior distribution to a parameter. As an example, when it is assumed that an error following a normal distribution having an average of 0 and a standard deviation σ is added to the model expressed by the formula (5) at the time of measurement, the model is represented as follows.

$$[ Formula 6 ]$$

$$β ( {t}_{i} , {T}_{i} , {H}_{i} ) ∼ 𝒩 ( \hat{β} ( {t}_{i} , {T}_{i} , {H}_{i} ) , σ ) ( i = 1 , 2 , … , L ) \qquad ( 6 )$$

$$where$$

$$\hat{β} ( t , T , H ) = {\int }_{0}^{t} k ( T , H ) \cdot {β}_{∞} {( \frac{\hat{β} ( {t}^{′} , T , H )}{{β}_{∞}} )}^{m} {( 1 - \frac{\hat{β} ( {t}^{′} , T , H )}{{β}_{∞}} )}^{n} {dt}^{′} + {β}_{0}$$

$$k ( T , H ) = A exp ( - \frac{E}{RT} + BH )$$

Here, N (μ, σ) represents a normal distribution having an average μ and a standard deviation σ. By providing a prior distribution to the parameters A, E, B, m, n, β₀, β_{∞} and σ and performing Bayesian inference by using MCMC (Markov chain Monte Carlo method), it is possible to derive the posterior distribution of each parameter or the posterior distribution of a peak area ratio β with respect to any value for each of t, T and H.

### (3-2) First Problem

The inventors of the present application have found that the basic concept of the estimation processing described in the above-mentioned (3-1) has two problems that make it difficult to make proper estimation. The first problem is that proper estimation is difficult when errors in regard to a plurality of acceleration factors are taken into consideration. Pharmaceuticals are produced through complicated processes, and there are various error factors other than a measurement noise. An error in regard to the acceleration factor include “variations in temperature” or “variations in humidity.” In a stability test, a pharmaceutical is stored in a space maintained at constant temperature and humidity, such as a stability test device or a stability test room. However, in such a test device, the temperature and/or humidity may change. Therefore, reaction may proceed faster or slower compared to a case of an ideal storage condition. In a case in which these errors are not incorporated into a model formula, it is assumed that pharmaceuticals stored in all testing devices are in the same condition. However, the data affected by the temperature or humidity are actually measured according to the storage environments. As a result, a measurement error may be estimated overly largely (overdispersion).

In order to deal with this problem, a method of providing, to a model formula, a hierarchical error term with respect to a temperature or a humidity for each sample stored in the same test device is considered. For example, when it is assumed that an error distribution of a temperature and a humidity of each test device follows a normal distribution having an average 0 and standard deviations σ_{T} and σ_{H}, a peak area ratio β can be modeled as in the formula (7).

$$[ Formula 7 ]$$

$$β ( {t}_{i} , {T}_{i} , {H}_{i} , {c}_{i} ) ∼ 𝒩 ( \hat{β} ( {t}_{i} , {T}_{i} , {H}_{i} , {c}_{i} ) , σ ) ( i = 1 , 2 , … , L ) \qquad ( 7 )$$

$$where$$

$$\hat{β} ( t , T , H , c ) = {\int }_{0}^{t} k ( T , H , c ) \cdot {β}_{∞} {( \frac{\hat{β} ( {t}^{′} , T , H , c )}{{β}_{∞}} )}^{m} {( 1 - \frac{\hat{β} ( {t}^{′} , T , H , c )}{{β}_{∞}} )}^{n} {dt}^{′} + {β}_{0}$$

$$k ( T , H , c ) = A exp ( - \frac{E}{R ( T + Δ {T}_{c} )} + B ( H + Δ {H}_{c} ) )$$

$$Δ {T}_{c} ∼ 𝒩 ( 0 , {σ}_{T} ) ( c ∈ 𝒞 = \{ 1 , 2 , … , \vert 𝒞 \vert \} )$$

$$Δ {H}_{c} ∼ 𝒩 ( 0 , {σ}_{H} ) ( c ∈ 𝒞 = \{ 1 , 2 , … , \vert 𝒞 \vert \} )$$

Here, C represents the collection of labels of test devices. Cᵢ indicates the label of the test device storing a pharmaceutical sample that is measured the i-th. With the formula (7), for each pharmaceutical sample, a temperature error and a humidity error according to the storing test device are taken into consideration.

However, when Bayesian inference is to be performed on the model expressed by the formula (7) with use of MCMC, a calculation period of time is significantly increased compared to the model expressed by the formula (6). Alternatively, an appropriate posterior distribution might not be obtained. It is considered that one of the causes is a problem depending on the method of MCMC and the shape of a likelihood function.

Although there are many methods of MCMC, a method of MCMC using a gradient of likelihood and represented by HMC (Hamiltonian Monte Carlo) or NUTS (No-U-Turn Sampler) is often used in recent years due to its computational efficiency. With these MCMC methods with use of a gradient of likelihood, a next point is searched or sampled with use of a result obtained when the obtained gradient is multiplied by a step size. When a step size is set in accordance with the scale of a gradient, there is an effect of preventing degradation of efficiency caused by a point to be searched being too far away or too close. A step size may be determined manually or may be determined automatically.

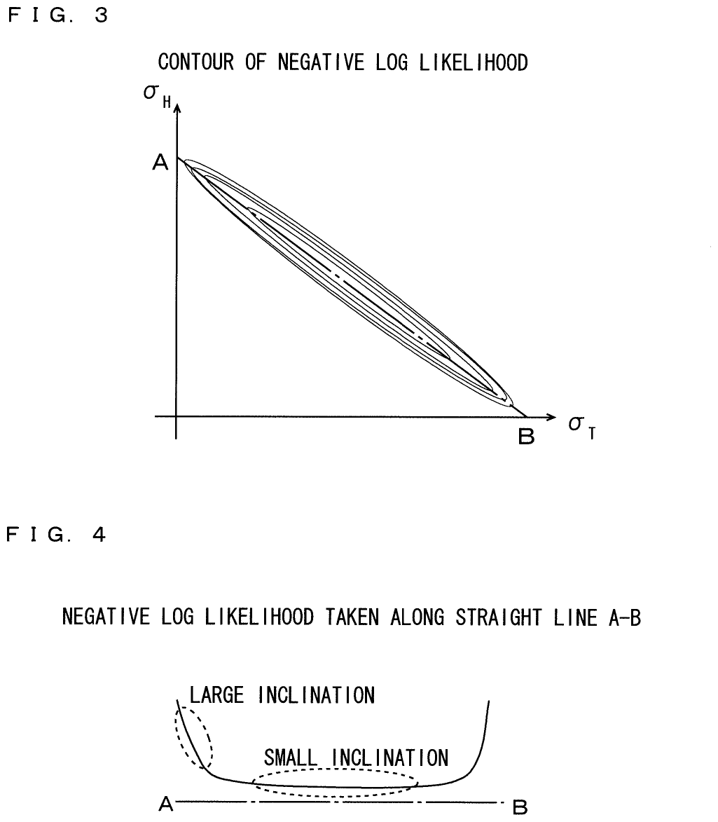

Here, a likelihood function in the present embodiment will be considered. What is measured in the present embodiment is a peak area ratio. While the temperature and humidity in each test device are set to predetermined values, the actual temperature or the actual humidity is not measured. Therefore, in a case in which reaction in a certain test device is fast compared to a case of an ideal condition, it is difficult to know whether it is caused by a difference in temperature or a difference in humidity. Because of this, the likelihood function focusing on a σ_{T} axis and a σ_{H} axis (with other parameters fixed) can take a shape in which σ_{T} and σ_{H} have a trade-off relationship as shown in FIG. 3. FIG. 3 shows the contour of a three-dimensional graph obtained when a negative log likelihood is plotted with respect to the σ_{T} and σ_{H} axes. FIG. 4 shows the cross section of the three-dimensional graph taken along the line A-B of FIG. 3. As shown in FIG. 4, the negative log likelihood has a bathtub shape in which a portion having “large inclination” and a portion having “small inclination” are mixed.

At this time, when a step size is set in accordance with the portion having large inclination, search efficiency is degraded in the portion having small inclination. Conversely, when a step size is set in accordance with the portion having small inclination, search efficiency is degraded in the portion having large inclination. In this manner, search efficiency or sampling efficiency is degraded due to no setting of a step size that works for the two regions. As a result, because the number of iterations required for sufficient search is significantly increased, there is a problem that an estimation period of time is long or appropriate estimation cannot be made. Also with a regression method other than Bayesian inference, a similar problem may occur in determination of a step size with a method using a gradient of likelihood.

### (3-3) Second Problem

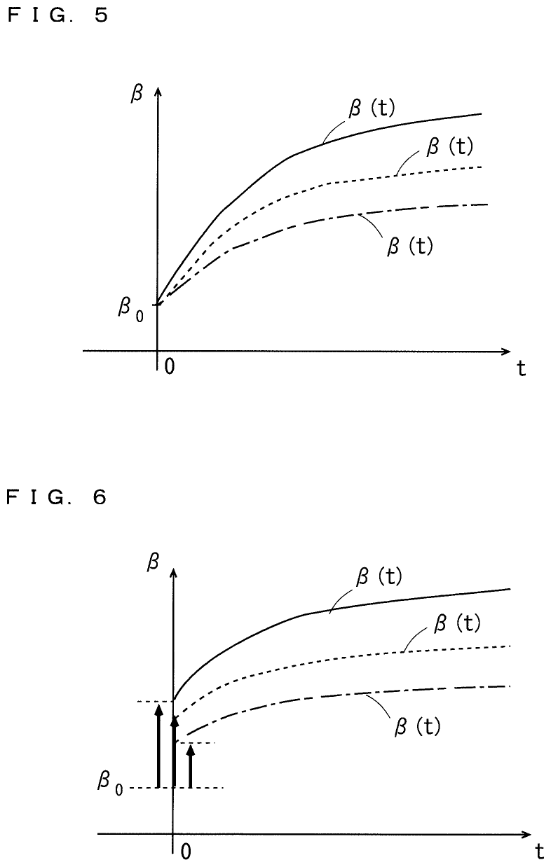

A second problem in regard to the basic concept of the estimation processing described in the above-mentioned (3-1) is that, in a case in which decomposition additionally occurs immediately after the start of storage, estimation cannot be made well with the model expressed by the formula (6). In a case in which no additional decomposition occurs immediately after the start of storage, the temporal change of a peak area ratio β(t) is as shown in FIG. 5 with an initial value obtained when t=0 being β₀.

In contrast, FIG. 6 shows the temporal change of the peak area ratio β(t) in a case in which additional decomposition occurs immediately after the start of storage. For example, it may include a case in which reaction proceeds fast in the vicinity of the surface of a pharmaceutical being in contact with air at the start of storage. Further, an acceleration condition such as a temperature or a humidity affects this additional reaction.

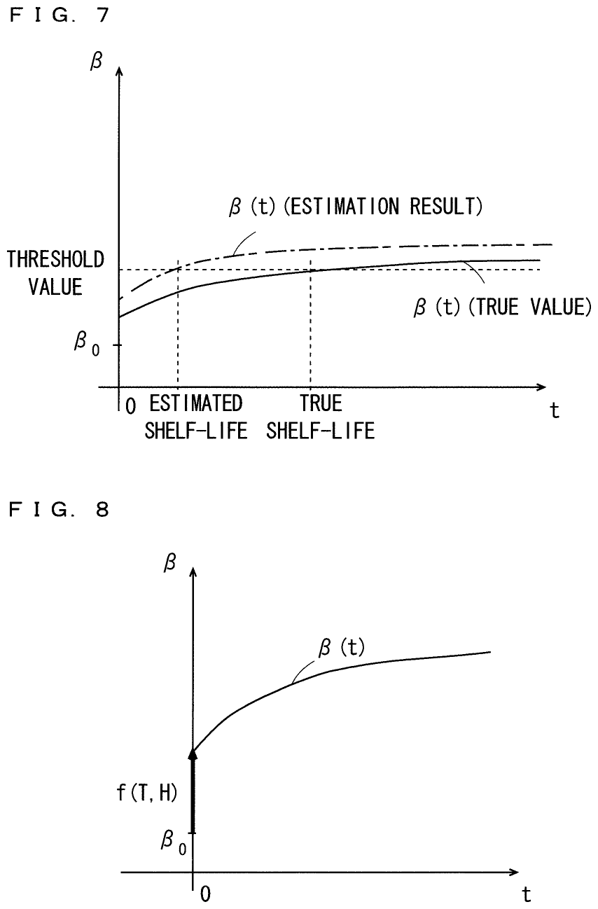

In a case in which the model expressed by the formula (6) that does not take an additional reaction into consideration is used with respect to the measurement data obtained with such an additional reaction, the peak area ratio of a pharmaceutical under a long-term storage condition is estimated as shown in FIG. 7. That is, the initial value of the peak area ratio is estimated to be excessively high due to an additional reaction. When a period (shelf-life) during which the peak area ratio does not exceed a threshold value is obtained based on this estimation result, the shelf-life is estimated to be excessively short.

### (3-4) Solution to First Problem

As the solution to the first problem of the above-mentioned (3-2), the estimator 23 of the present embodiment performs the following estimation processing. The estimator 23 does not set an error distribution for each acceleration factor but sets an error distribution with respect to a reaction rate constant k obtained when these acceleration factors are integrated. That is, instead of setting of an individual error distribution with respect to each of the temperature and humidity for the model formula as shown in the formula (7), one error distribution for integration of the temperature and the humidity is set for the model formula. That is, the estimator 23 uses the reaction model RM expressed by the formula (8) instead of the formula (7).

$$[ Formula 8 ]$$

$$β ( {t}_{i} , {T}_{i} , {H}_{i} , {c}_{i} ) ∼ 𝒩 ( \hat{β} ( {t}_{i} , {T}_{i} , {H}_{i} , {c}_{i} ) , σ ) ( i = 1 , 2 , … , L ) \qquad ( 8 )$$

$$where$$

$$\hat{β} ( t , T , H , c ) = {\int }_{0}^{t} k ( T , H , c ) \cdot {β}_{∞} {( \frac{\hat{β} ( {t}^{′} , T , H )}{{β}_{∞}} )}^{m} {( 1 - \frac{\hat{β} ( {t}^{′} , T , H )}{{β}_{∞}} )}^{n} {dt}^{′} + {β}_{0}$$

$$k ( T , H , c ) = A exp ( - \frac{E}{RT} + BH ) + Δ {k}_{c}$$

$$Δ {k}_{c} ∼ 𝒩 ( 0 , {σ}_{k} ) .$$

Thus, the above-mentioned trade-off relationship between σ_{T} and σ_{H} is dissolved, and a problem caused by a bathtub shape of a likelihood function is suppressed. Data defining the reaction model expressed by the formula (8) is stored in the storage device 16 as the reaction model RM. The model formula expressed by the formula (8) is generated based on the solid reaction model expressed by the formula (2) and is an example. Based on another solid reaction model, an error distribution for integrated acceleration factors may be set similarly to the formula (8). A plurality of reaction models RM may be stored in the storage device 16, and one of them may be selected and used. In this manner, by using the model formula in which an error distribution for integration of the acceleration factors is set, it is possible to alleviate a problem in regard to a step size caused by a bathtub shape, described above, and acquire a more appropriate and useful estimation result that takes an error in regard to the acceleration factors into account in a practical period of time.

### (3-5) Solution to the Second Problem

As the solution to the second problem of the above-mentioned (3-3), the estimator 23 of the present embodiment performs the following estimation processing. The estimator 23 uses a model formula that assumes an additional reaction in accordance with an acceleration condition. As a method of modeling an additional reaction, two methods are further considered. There are a method of modeling an additional reaction as it is and a method of considering an additional reaction as a difference from a true point in time and modeling it as a point-in-time offset.

### (3-5-1) Modeling of Additional Reaction

Of the two methods, the method of modeling an additional reaction is as follows. The estimator 23 uses the reaction model RM expressed by the formula (9) obtained when an additional reaction is added to the model formula expressed by the formula (6).

$$[ Formula 9 ]$$

$$β ( {t}_{i} , {T}_{i} , {H}_{i} ) ∼ 𝒩 ( \hat{β} ( {t}_{i} , {T}_{i} , {H}_{i} ) , σ ) ( i = 1 , 2 , … , N ) \qquad ( 9 )$$

$$where$$

$$\hat{β} ( t , T , H ) = {\int }_{0}^{t} k ( T , H ) \cdot {β}_{∞} {( \frac{\hat{β} ( {t}^{′} , T , H )}{{β}_{∞}} )}^{m} {( 1 - \frac{\hat{β} ( {t}^{′} , T , H )}{{β}_{∞}} )}^{n} {dt}^{′} + {β}_{0} + f ( T , H )$$

$$k ( T , H ) = A exp ( - \frac{E}{RT} + BH )$$

In the formula (9), f is a function representing an additional reaction. The conceptual diagram of the additional reaction function f is shown in FIG. 8. For example, in a case in which an additional reaction linearly occurs with respect to a reaction rate constant k, that is, a case in which f(T, H)=x·k(T, H) is used as a parameter representing the magnitude of the additional reaction, a model formula is expressed by the formula (10).

$$[ Formula 10 ]$$

$$β ( {t}_{i} , {T}_{i} , {H}_{i} ) ∼ 𝒩 ( \hat{β} ( {t}_{i} , {T}_{i} , {H}_{i} ) , σ ) ( i = 1 , 2 , … , N ) \qquad ( 10 )$$

$$where$$

$$\hat{β} ( t , T , H ) = {\int }_{0}^{t} k ( T , H ) \cdot {β}_{∞} {( \frac{\hat{β} ( {t}^{′} , T , H )}{{β}_{∞}} )}^{m} {( 1 - \frac{\hat{β} ( {t}^{′} , T , H )}{{β}_{∞}} )}^{n} {dt}^{′} + {β}_{0} + x \cdot k ( T , H )$$

$$k ( T , H ) = A exp ( - \frac{E}{RT} + BH )$$

Data defining the reaction model expressed by the formula (9) and the formula (10) is stored in the storage device 16 as the reaction model RM. The model formulas expressed by the formula (9) and the formula (10) are generated based on the solid reaction model represented by the formula (2), and are examples. Based on another solid reaction models, additional reactions may be added similarly to the formula (9) and the formula (10). A plurality of reaction models RM may be stored in the storage device 16, and one of them may be selected and used. In this manner, when an additional reaction is modeled, even in a case in which there is an additional reaction, an estimation result obtained with the additional reaction taken into account is acquired, and the shelf-life can be prevented from being estimated to be excessively short.

### (3-5-2) Modeling of Point-In-Time Offset

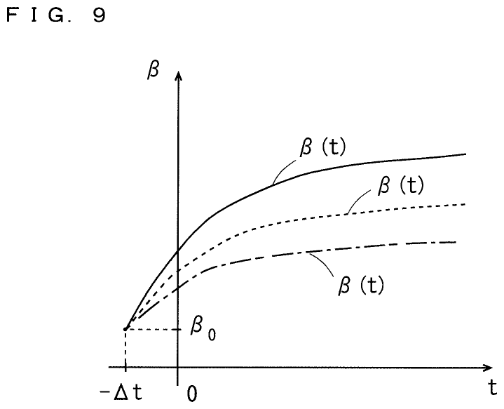

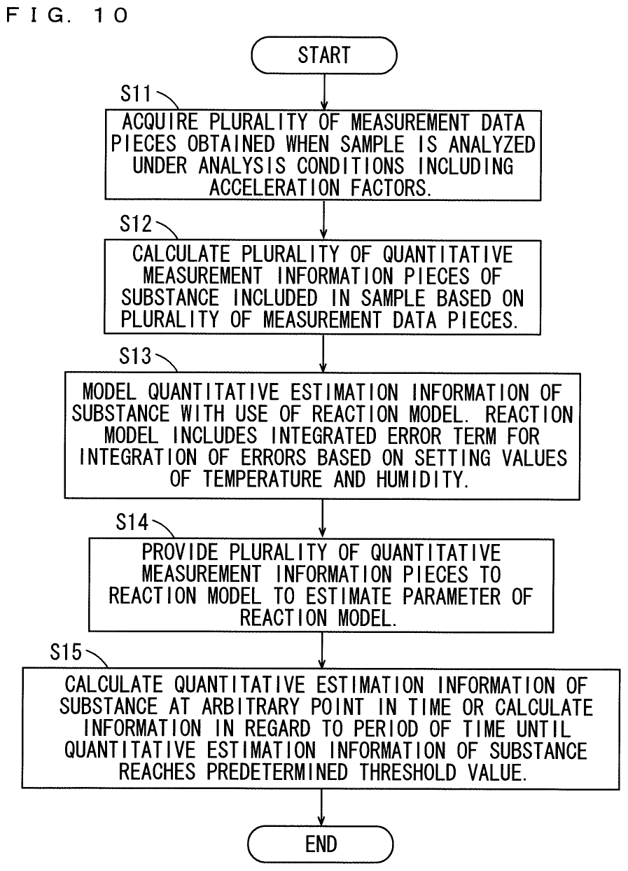

Of the two methods, the method of modeling a point-in-time offset is as follows. The estimator 23 introduces an offset term Δt with respect to a point in time into the model formula expressed by the formula (6) and uses the reaction model RM expressed by the formula (11). FIG. 9 is the conceptual diagram of the offset term Δt.

$$[ Formula 11 ]$$

$$β ( {t}_{i} , {T}_{i} , {H}_{i} ) ∼ 𝒩 ( \hat{β} ( {t}_{i} , {T}_{i} , {H}_{i} ) , σ ) ( i = 1 , 2 , … , N ) \qquad ( 11 )$$

$$where$$

$$\hat{β} ( t , T , H ) = {\int }_{- Δ t}^{t} k ( T , H ) \cdot {β}_{∞} {( \frac{\hat{β} ( {t}^{′} , T , H )}{{β}_{∞}} )}^{m} {( 1 - \frac{\hat{β} ( {t}^{′} , T , H )}{{β}_{∞}} )}^{n} {dt}^{′} + {β}_{0}$$

$$k ( T , H ) = A exp ( - \frac{E}{RT} + BH )$$

Data defining the reaction model expressed by the formula (11) is stored in the storage device 16 as the reaction model RM. The model formula expressed by the formula (11) is generated based on the solid reaction model expressed by the formula (2) and is an example. Based on another solid reaction model, the offset term Δt may be set similarly to the formula (11). A plurality of reaction models RM may be stored in the storage device 16, and one of them may be selected and used. In this manner, when a time difference in regard to the start of an analysis is set, even in a case in which there is an additional reaction, an estimation result obtained when the additional reaction is appropriately taken into account is acquired, and the shelf-life can be prevented from being estimated to be excessively short.

### (4) Sample Analysis Method

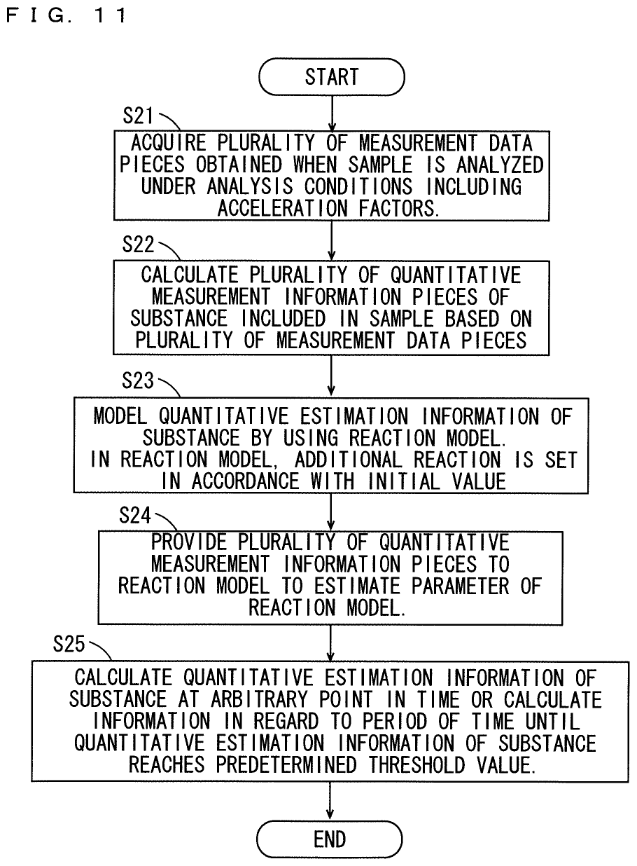

Next, a sample analysis method according to an embodiment will be described with reference to the flowcharts of FIGS. 10 to 12. The flowcharts of FIGS. 10 to 12 show a process to be executed by the CPU 11 shown in FIG. 1. That is, the process is to be executed by each of the functions 21 to 24 shown in FIG. 2 when the CPU 11 runs the program P1 while using the hardware resources such as the RAM 12.

First, with reference to FIG. 10, the sample analysis method based on the above-mentioned (3-4) Solution to First Problem will be described. In the step S11, the acquirer 21 acquires a plurality of measurement data pieces MD obtained when a sample is analyzed in an analysis device under a plurality of analysis conditions including a temperature and a humidity as acceleration factors. That is, the measurement data MD is the data obtained based on a sample (pharmaceutical) stored under conditions severer than a normal storage environment in terms of the temperature and humidity.

Next, in the step S12, the quantitative information calculator 22 calculates a plurality of quantitative measurement information pieces of a substance included in the sample based on the plurality of measurement data pieces MD. In the embodiment, the quantitative information calculator 22 calculates peak area ratio data PS (the ratio of a peak area of impurities with respect to a peak area of an active ingredient included in the pharmaceutical).

Next, in the step S13, the estimator 23 retrieves a reaction model ML stored in the storage device 16 and models the quantitative estimation information of the substance with use of the reaction model RM. Here, as shown in the formula (8), the reaction model RM includes an integrated error term for integration of errors based on set values of the temperature and humidity.

Next, in the step S14, the estimator 23 provides the plurality of quantitative measurement information pieces calculated by the quantitative information calculator 22 to the reaction model RM to estimate a parameter PM of the reaction model RM. For example, the estimator 23 performs a regression analysis such as Bayesian inference, a least squares method or MAP estimation to estimate the parameter PM of the reaction model RM.

Next, in the step S15, the calculator 24 calculates the quantitative estimation information of the substance at an arbitrary point in time based on the parameter estimated by the estimator 23. In an example of the present embodiment, the calculator 24 calculates an estimation value of the peak area ratio (the ratio of a peak area of impurities with respect to a peak area of an active ingredient included in a pharmaceutical) at an arbitrary point in time (the number of days). Alternatively, the calculator 24 calculates the information in regard to a period of time until the quantitative estimation information of the substance reaches a predetermined threshold value. In the example of the embodiment, the calculator 24 calculates a period of time (the number of days) until an estimation value of the peak area ratio (the ratio of a peak area of impurities with respect to a peak area of an active ingredient included in a pharmaceutical) reaches a predetermined value. The outputter 25 outputs the estimation result calculated by the calculator 24 to the display 15.

Further, in a case in which the estimator 23 uses a regression analysis method of estimating a posterior distribution such as Bayesian inference, the calculator 24 calculates a confidence interval or a quantile of a quantitative value of a substance at an arbitrary point in time. Alternatively, the calculator 24 calculates a confidence interval and a quantile in a period of time until the quantitative estimation information of the substance reaches the predetermined threshold value.

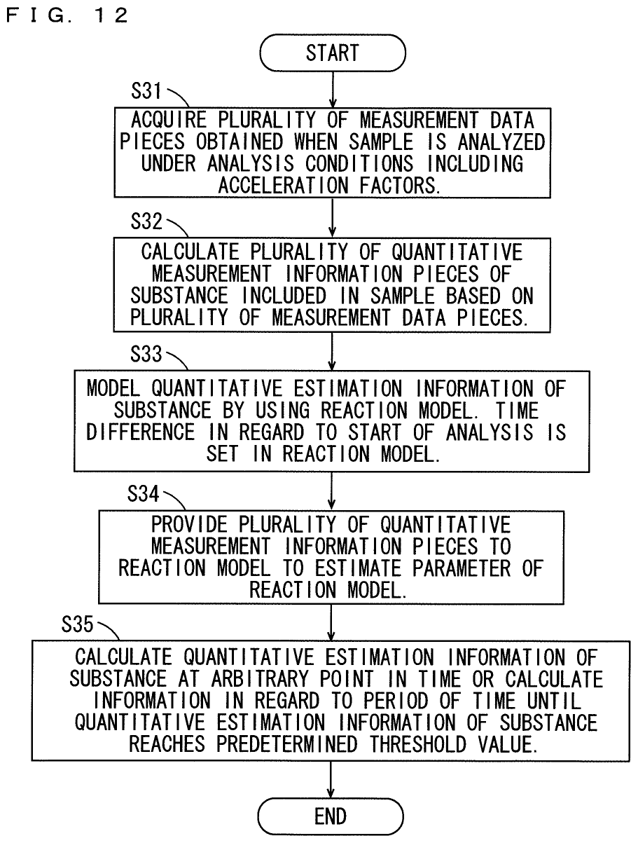

Next, the sample analysis method based on the above-mentioned (3-5-1) Solution to Second Problem (Modeling of Additional Reaction) will be described with reference to FIG. 11. Because the process of the steps S21 and S22 is similar to the steps S11 and S12 in FIG. 10, a description thereof is not repeated.

Next, in the step S23, the estimator 23 retrieves the reaction model ML stored in the storage device 16 and models the quantitative estimation information of the substance by using the reaction model RM. Here, in the reaction model RM, an additional reaction is set in accordance with an initial value as shown in the formulas (9) and (10). Subsequently, because the steps S24 and S25 are similar to the steps S14 and S15 in FIG. 10, a description thereof will not be repeated.

Next, with reference to FIG. 12, the sample analysis method based on the above-mentioned (3-5-2) Solution to Second Problem (Modeling of Point-In-Time Offset) will be described. Because the steps S31 and S32 are similar to the steps S11 and S12 of FIG. 10, a description thereof will not be repeated.

In the step S33, the estimator 23 retrieves the reaction model ML stored in the storage device 16 and models the quantitative estimation information of the substance by using the reaction model RM. Here, as shown in the formula (11), the offset term Δt representing a time difference in regard to the start of an analysis is set in the reaction model RM. Subsequently, because the steps S34 and S35 are similar to the steps S14 and S15 of FIG. 10, a description thereof will not be repeated.

### (5) Simulation Results

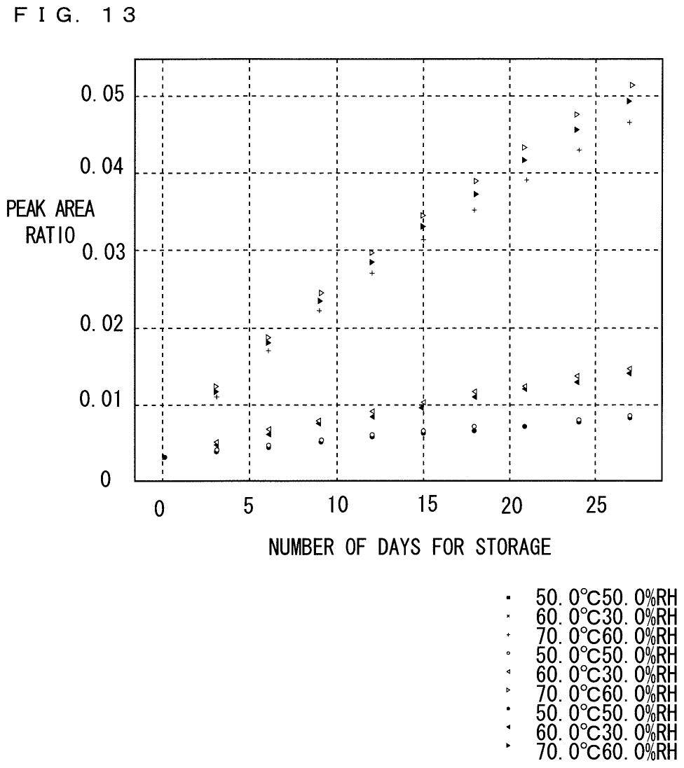

Simulation results of the estimation processing in the present embodiment will be described. As an example, it is assumed that the simulation data as shown in FIG. 13 is obtained in regard to the peak area ratio of a sample stored with variations in temperature and humidity. In FIG. 13, the abscissa indicates the number of days for storage, and the ordinate indicates the peak area ratio with respect to a main component. The diagram shows the data obtained for storage of about 30 days under an acceleration condition. Here, the legends including different symbols indicate that it is the data of samples stored in different test devices. For example, although there are three types of symbols for 70° C. 60% RH, each of them has a slightly different reaction rate. This indicates that, even with the same set value of 70° C. 60% RH, there were variations in temperature and humidity depending on the test devices, and there were differences in peak area ratio. Similarly, simulation is performed in regard to three types of data with variations with respect to the set values of 50° C. 50% RH and 60° C. 30% RH.

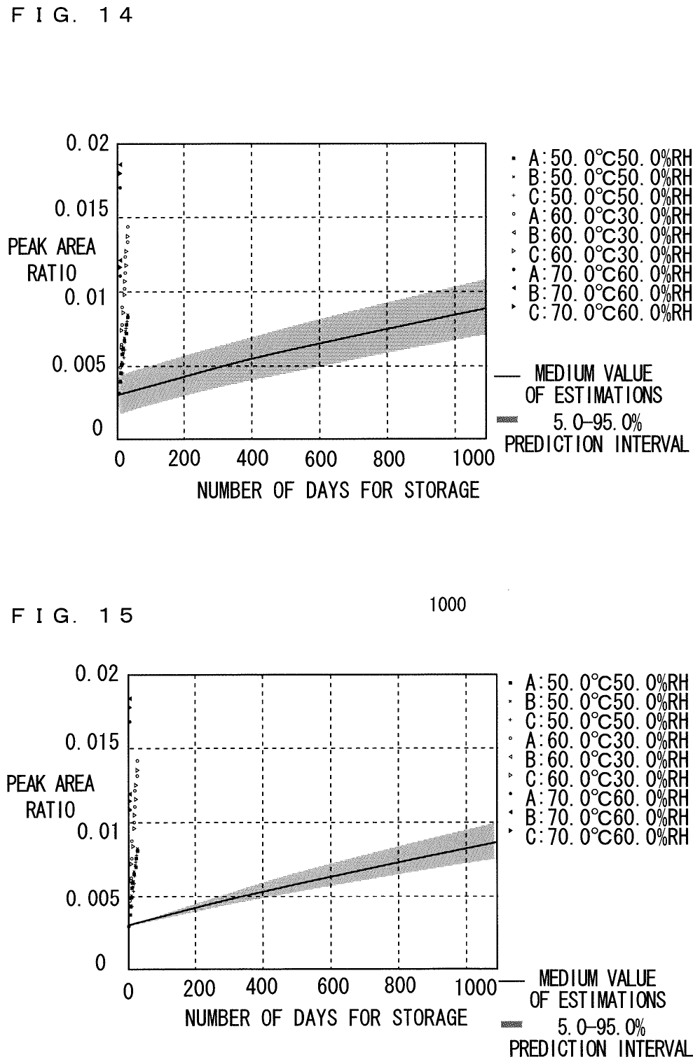

With use of the simulation data, FIG. 14 shows the prediction interval for 25° C. 60% RH obtained by the model formula expressed by the formula (6). In FIG. 14, the abscissa indicates the number of days for storage, and the ordinate indicates the peak area ratio with respect to the main component. In FIG. 14, the thick solid line indicates the median value of estimation, the hatched region indicates the 90% prediction interval of estimation, and the symbols indicate the data used for estimation (data shown in FIG. 13). The peak area ratio after storage of about 1000 days is estimated based on the simulation data for about 30 days.

In contrast, with use of the simulation data, FIG. 15 shows the prediction interval for 25° C. 60% RH obtained by the model formula, into which a hierarchical error is introduced, expressed by the formula (8). In FIG. 15, the abscissa indicates the number of days for storage, and the ordinate indicates the peak area ratio of the main component. Similarly, the peak area ratio after storage for about 1000 days is estimated based on the simulation data for about 30 days. In FIG. 15, the thick solid line indicates the median value of estimation, the hatched region indicates the 90% prediction interval of estimation, and the symbols indicate the data used for estimation (data shown in FIG. 13). When FIG. 14 and FIG. 15 are compared with each other, it can be seen that the model formula into which the hierarchical error is introduced provides the narrower prediction interval.

### (6) Other Embodiments

In each above-mentioned embodiment, the sample analysis device 1 analyzes a pharmaceutical as a sample, by way of example. The sample analysis device 1 of the present embodiment can be utilized to acquire the quantitative estimation information of a substance in various samples other than pharmaceuticals. In the above-mentioned embodiment, the peak area ratio is an example of quantitative estimation information of a substance, by way of example. The sample analysis device 1 of the present invention can also estimate a peak area, a retention time and the like as the quantitative estimation information of a substance.

In the above-mentioned embodiment, the temperature and humidity are used as acceleration factors, by way of example. In addition, light may be used as the acceleration factor. For example, the temperature and light, the humidity and light, or the like can be used as the acceleration factors.

### (7) Aspects

It will be appreciated by those skilled in the art that the exemplary embodiments described above are illustrative of the following aspects.

(Item 1) A sample analysis device according to one aspect includes an acquirer that acquires a plurality of measurement data pieces obtained by an analysis of a sample using an analysis device under a plurality of analysis conditions, the analysis conditions including a temperature and a humidity as acceleration factors, a quantitative information calculator that calculates, based on the plurality of measurement data pieces, a plurality of quantitative measurement information pieces of a substance included in the sample, an estimator that retrieves a reaction model stored in a storage device, models quantitative estimation information of the substance with use of the reaction model, and provides the plurality of quantitative measurement information pieces calculated by the quantitative information calculator to the reaction model to estimate a parameter of the reaction model, and a calculator that calculates, based on the parameter estimated by the estimator, quantitative estimation information of the substance at an arbitrary point in time or calculates information in regard to a period of time until quantitative estimation information of the substance reaches a predetermined threshold value, wherein an integrated error term for integration of errors based on setting values of the temperature and the humidity set under the plurality of analysis conditions is included in the reaction model.

It is possible to suppress degradation of accuracy for estimation of a reaction model due to an error caused by an acceleration factor.

(Item 2) A sample analysis device according to another aspect includes an acquirer that acquires a plurality of measurement data pieces obtained by an analysis of a sample using an analysis device under a plurality of analysis conditions, the analysis conditions including a temperature and a humidity as acceleration factors, a quantitative information calculator that calculates, based on the plurality of measurement data pieces, a plurality of quantitative measurement information pieces of a substance included in the sample, an estimator that retrieves a reaction model stored in a storage device, models quantitative estimation information of the substance with use of the reaction model, and provides the plurality of quantitative measurement information pieces calculated by the quantitative information calculator to the reaction model to estimate a parameter of the reaction model; and a calculator that calculates, based on the parameter estimated by the estimator, quantitative estimation information of the substance at an arbitrary point in time or calculates information in regard to a period of time until quantitative estimation information of the substance reaches a predetermined threshold value, wherein an additional reaction is set in the reaction model in accordance with an initial value.

Even in a case in which there is an additional reaction, an estimation result obtained when the additional reaction is appropriately taken into consideration is acquired.

(Item 3) A sample reaction device according to another aspect includes an acquirer that acquires a plurality of measurement data pieces obtained by an analysis of a sample using an analysis device under a plurality of analysis conditions, the analysis conditions including a temperature and a humidity as acceleration factors, a quantitative information calculator that calculates, based on the plurality of measurement data pieces, a plurality of quantitative measurement information pieces of a substance included in the sample, an estimator that retrieves a reaction model stored in a storage device, models quantitative estimation information of the substance with use of the reaction model, and provides the plurality of quantitative measurement information pieces calculated by the quantitative information calculator to the reaction model to estimate a parameter of the reaction model, and a calculator that calculates, based on the parameter estimated by the estimator, quantitative estimation information of the substance at an arbitrary point in time or calculates information in regard to a period of time until quantitative estimation information of the substance reaches a predetermined threshold value, wherein a time difference in regard to start of an analysis is set in the reaction model.

Even in a case in which there is an additional reaction, an estimation result obtained when the additional reaction is appropriately taken into consideration is acquired.

(Item 4) The sample analysis device according to anyone of items 1 to 3, wherein an Arrhenius equation or a modified Arrhenius equation may be applied to the reaction model.

Quantitative estimation information in regard to any temperature, any humidity and an arbitrary point in time is obtained.

(Item 5) The sample analysis device according to any one of items 1 to 3, wherein light may be included as the acceleration factor.

The accuracy of modeling is also improved for a reaction with light as an acceleration factor.

(Item 6) The sample analysis device according to any one of items 1 to 3, wherein a plurality of reaction models may be stored in the storage device.

An appropriate solid reaction model can be used.

(Item 7) The sample analysis device according to any one of items 1 to 3, wherein quantitative estimation information of the substance may include a quantitative value, a confidence interval or a quantile of the substance at an arbitrary point in time.

Quantitative estimation information enables various analyses.

(Item 8) The sample analysis device according to any one of items 1 to 3, wherein information in regard to the period of time may include a value, a confidence interval or a quantile in a period of time until quantitative estimation information of the substance reaches a predetermined threshold value.

Quantitative estimation information enables various analyses.

(Item 9) In the sample analysis device according to any one of items 1 to 3, the sample may include a formulation or a drug substance, and the substance may include an active ingredient or an impurity present in the formulation or the drug substance.

A component of a pharmaceutical can be analyzed with high accuracy.

(Item 10) A sample analysis method according to another aspect includes acquiring a plurality of measurement data pieces obtained by an analysis of a sample using an analysis device under a plurality of analysis conditions, the analysis conditions including a temperature and a humidity as acceleration factors, calculating, based on the plurality of measurement data pieces, a plurality of quantitative measurement information pieces of a substance included in the sample, retrieving a reaction model stored in a storage device, modelling quantitative estimation information of the substance with use of the reaction model, and providing the plurality of quantitative measurement information pieces to the reaction model to estimate a parameter of the reaction model, and calculating, based on the estimated parameter, quantitative estimation information of the substance at an arbitrary point in time or calculating information in regard to a period of time until quantitative estimation information of the substance reaches a predetermined threshold value, wherein an integrated error term for integration of errors based on setting values of the temperature and the humidity set under the plurality of analysis conditions is included in the reaction model.

It is possible to suppress degradation of accuracy for estimation of a reaction model due to an error caused by an acceleration factor.

(Item 11) A sample analysis method according to another aspect includes acquiring a plurality of measurement data pieces obtained by an analysis of a sample using an analysis device under a plurality of analysis conditions, the analysis conditions including a temperature and a humidity as acceleration factors, calculating, based on the plurality of measurement data pieces, a plurality of quantitative measurement information pieces of a substance included in the sample, retrieving a reaction model stored in a storage device, modelling quantitative estimation information of the substance with use of the reaction model, and providing the plurality of quantitative measurement information pieces to the reaction model to estimate a parameter of the reaction model, and calculating, based on the estimated parameter, quantitative estimation information of the substance at an arbitrary point in time or calculating information in regard to a period of time until quantitative estimation information of the substance reaches a predetermined threshold value, wherein an additional reaction is set in the reaction model in accordance with an initial value.

Even in a case in which there is an additional reaction, an estimation result obtained when the additional reaction is appropriately taken into consideration is acquired.

(Item 12) A sample analysis method according to another aspect includes acquiring a plurality of measurement data pieces obtained by an analysis of a sample using an analysis device under a plurality of analysis conditions, the analysis conditions including a temperature and a humidity as acceleration factors, calculating, based on the plurality of measurement data pieces, a plurality of quantitative measurement information pieces of a substance included in the sample, retrieving a reaction model stored in a storage device, modeling quantitative estimation information of the substance with use of the reaction model, and providing the plurality of quantitative measurement information pieces to the reaction model to estimate a parameter of the reaction model, and calculating, based on the estimated parameter, quantitative estimation information of the substance at an arbitrary point in time or calculating information in regard to a period of time until quantitative estimation information of the substance reaches a predetermined threshold value, wherein a time difference in regard to start of an analysis is set in the reaction model.

Even in a case in which there is an additional reaction, an estimation result obtained when the additional reaction is appropriately taken into consideration is acquired.

(Item 13) In the sample analysis method according to any one of items 10 to 12, the sample may include a formulation or a drug substance, and the substance may include an active ingredient or an impurity present in the formulation or the drug substance.

A component of a pharmaceutical can be analyzed with high accuracy.

While preferred embodiments of the present disclosure have been described above, it is to be understood that variations and modifications will be apparent to those skilled in the art without departing the scope and spirit of the present disclosure. The scope of the present disclosure, therefore, is to be determined solely by the following claims.

I/We claim:

1. A sample analysis device comprising:
an acquirer that acquires a plurality of measurement data pieces obtained by an analysis of a sample using an analysis device under a plurality of analysis conditions, the analysis conditions including a temperature and a humidity as acceleration factors;
a quantitative information calculator that calculates, based on the plurality of measurement data pieces, a plurality of quantitative measurement information pieces of a substance included in the sample;
an estimator that
retrieves a reaction model stored in a storage device,
models quantitative estimation information of the substance with use of the reaction model, and
provides the plurality of quantitative measurement information pieces calculated by the quantitative information calculator to the reaction model to estimate a parameter of the reaction model; and
a calculator that calculates, based on the parameter estimated by the estimator, quantitative estimation information of the substance at an arbitrary point in time or calculates information in regard to a period of time until quantitative estimation information of the substance reaches a predetermined threshold value, wherein
an integrated error term for integration of errors based on setting values of the temperature and the humidity set under the plurality of analysis conditions is included in the reaction model.

2. A sample analysis device comprising:
an acquirer that acquires a plurality of measurement data pieces obtained by an analysis of a sample using an analysis device under a plurality of analysis conditions, the analysis conditions including a temperature and a humidity as acceleration factors;
a quantitative information calculator that calculates, based on the plurality of measurement data pieces, a plurality of quantitative measurement information pieces of a substance included in the sample;
an estimator that
retrieves a reaction model stored in a storage device,
models quantitative estimation information of the substance with use of the reaction model, and
provides the plurality of quantitative measurement information pieces calculated by the quantitative information calculator to the reaction model to estimate a parameter of the reaction model; and
a calculator that calculates, based on the parameter estimated by the estimator, quantitative estimation information of the substance at an arbitrary point in time or calculates information in regard to a period of time until quantitative estimation information of the substance reaches a predetermined threshold value, wherein
an additional reaction is set in the reaction model in accordance with an initial value.

3. A sample reaction device, comprising:
an acquirer that acquires a plurality of measurement data pieces obtained by an analysis of a sample using an analysis device under a plurality of analysis conditions, the analysis conditions including a temperature and a humidity as acceleration factors;
a quantitative information calculator that calculates, based on the plurality of measurement data pieces, a plurality of quantitative measurement information pieces of a substance included in the sample;
an estimator that
retrieves a reaction model stored in a storage device,
models quantitative estimation information of the substance with use of the reaction model, and
provides the plurality of quantitative measurement information pieces calculated by the quantitative information calculator to the reaction model to estimate a parameter of the reaction model; and
a calculator that calculates, based on the parameter estimated by the estimator, quantitative estimation information of the substance at an arbitrary point in time or calculates information in regard to a period of time until quantitative estimation information of the substance reaches a predetermined threshold value, wherein
a time difference in regard to start of an analysis is set in the reaction model.

4. The sample analysis device according to claim 1, wherein
an Arrhenius equation or a modified Arrhenius equation is applied to the reaction model.

5. The sample analysis device according to claim 1, wherein
light is included as the acceleration factor.

6. The sample analysis device according to claim 1, wherein
a plurality of reaction models are stored in the storage device.

7. The sample analysis device according to claim 1, wherein
quantitative estimation information of the substance includes a quantitative value, a confidence interval or a quantile of the substance at an arbitrary point in time.

8. The sample analysis device according to claim 1, wherein
information in regard to the period of time includes a value, a confidence interval or a quantile in a period of time until quantitative estimation information of the substance reaches a predetermined threshold value.

9. A pharmaceutical analysis device, wherein
in the sample analysis device according to claim 1, the sample includes a formulation or a drug substance, and the substance includes an active ingredient or an impurity present in the formulation or the drug substance.

10. A sample analysis method including:
acquiring a plurality of measurement data pieces obtained by an analysis of a sample using an analysis device under a plurality of analysis conditions, the analysis conditions including a temperature and a humidity as acceleration factors;
calculating, based on the plurality of measurement data pieces, a plurality of quantitative measurement information pieces of a substance included in the sample;
retrieving a reaction model stored in a storage device, modelling quantitative estimation information of the substance with use of the reaction model, and providing the plurality of quantitative measurement information pieces to the reaction model to estimate a parameter of the reaction model; and
calculating, based on the estimated parameter, quantitative estimation information of the substance at an arbitrary point in time or calculating information in regard to a period of time until quantitative estimation information of the substance reaches a predetermined threshold value, wherein
an integrated error term for integration of errors based on setting values of the temperature and the humidity set under the plurality of analysis conditions is included in the reaction model.

11. A sample analysis method including:
acquiring a plurality of measurement data pieces obtained by an analysis of a sample using an analysis device under a plurality of analysis conditions, the analysis conditions including a temperature and a humidity as acceleration factors;
calculating, based on the plurality of measurement data pieces, a plurality of quantitative measurement information pieces of a substance included in the sample;
retrieving a reaction model stored in a storage device, modelling quantitative estimation information of the substance with use of the reaction model, and providing the plurality of quantitative measurement information pieces to the reaction model to estimate a parameter of the reaction model; and
calculating, based on the estimated parameter, quantitative estimation information of the substance at an arbitrary point in time or calculating information in regard to a period of time until quantitative estimation information of the substance reaches a predetermined threshold value, wherein
an additional reaction is set in the reaction model in accordance with an initial value.

12. A sample analysis method including:
acquiring a plurality of measurement data pieces obtained by an analysis of a sample using an analysis device under a plurality of analysis conditions, the analysis conditions including a temperature and a humidity as acceleration factors;
calculating, based on the plurality of measurement data pieces, a plurality of quantitative measurement information pieces of a substance included in the sample;
retrieving a reaction model stored in a storage device, modelling quantitative estimation information of the substance with use of the reaction model, and providing the plurality of quantitative measurement information pieces to the reaction model to estimate a parameter of the reaction model; and
calculating, based on the estimated parameter, quantitative estimation information of the substance at an arbitrary point in time or calculating information in regard to a period of time until quantitative estimation information of the substance reaches a predetermined threshold value, wherein
a time difference in regard to start of an analysis is set in the reaction model.

13. A pharmaceutical analysis method, wherein
in the sample analysis device according to claim 10, the sample includes a formulation or a drug substance, and the substance includes an active ingredient or an impurity present in the formulation or the drug substance.
